(19) United States
(12) Patent Application Publication
(10) Pub. No.: US 2019/0233791 A1
(43) Pub. Date: Aug. 1, 2019

(54) MICROFLUIDIC DEVICE FOR STUDYING SHEAR STRESS AND TUMOR MIGRATION IN MICROCHANNEL AND METHOD OF ANALYZING CELLS USING THE MICROFLUIDIC DEVICE

(71) Applicant: INDUSTRY-UNIVERSITY COOPERATION FOUNDATION SOGANG UNIVERSITY, Seoul (KR)

(72) Inventors: Bong Geun CHUNG, Gyeonggi-do (KR); Da Yeon PARK, Busan (KR); Tae Hyeon KIM, Seoul (KR); Jong Min LEE, Seoul (KR); Christian Daniel Ahrberg, Seoul (KR)

(21) Appl. No.: 16/237,270

(22) Filed: Dec. 31, 2018

(30) Foreign Application Priority Data
Jan. 30, 2018 (KR) 10-2018-0011123

(51) Int. Cl. C12M 3/06; C12M 1/42; C12M 1/00; C12Q 1/02

(52) U.S. Cl. CPC C12M 23/16; C12Q 1/02; C12M 23/20; C12M 35/08

(57) ABSTRACT

A microfluidic device for studying shear stress and tumor migration in a microchannel includes: a microchamber; a microchannel including an inlet and an outlet for a fluid and formed on the periphery of the microchamber and making the fluid pass through the periphery of the microchamber; and a plurality of groups of bridge channels connecting the microchannel and the microchannel at a plurality of locations in the microchannel and a cross-sectional area of the bridge channel is smaller than that of the microchannel.

### CROSS-REFERENCE TO RELATED APPLICATION(S)

This application claims the benefit under 35 USC § 119(a) of Korean Patent Application No. 10-2018-0011123 filed on Jan. 30, 2018 in the Korean Intellectual Property Office, the entire disclosures of which are incorporated herein by reference for all purposes.

### TECHNICAL FIELD

The present invention relates to a microfluidic device, and more particularly, to a microfluidic device for studying shear stress and tumor migration in relation to cells in a microchannel and a method for analyzing cells using the microfluidic device.

### BACKGROUND

A blood vessel as a kind of microchannel may be a passage through which many cells and tumors pass. However, in the related art, it has been difficult to study an effect of shear stress and flow rate on the cell in terms of shear stress and tumor migration in the microchannel, and there are not many methods to precisely simulate them.

Korean Patent Registration No. 10-1370931 discloses a cell culture apparatus capable of analyzing cell responses to external stimuli in real time while independently controlling shear stress and an electric field. However, the registration patent also may be pointed out as a disadvantage in that the change of cells due to the shear stress can not be compared at a time.

### TECHNICAL SUBJECTS

The present invention provides a microfluidic device for studying shear stress and tumor migration and provides a microfluidic device capable of studying various phenomena by appropriately designing a channel and showing a gradient of the shear stress according to a plurality of zones.

The present invention provides a microfluidic device which is capable of examining an aspect ratio of cells according to the shear stress in each zone in the microfluidic device and is suitable for optimizing shear stress conditions.

The present invention provides a microfluidic device which can rapidly optimize conditions for various cells and can more precisely simulate phenomena which occur in the blood vessel because the shear stress and the tumor migration can be studied based on hydrogel by using one microfluidic device or microfluidic chip.

### SUMMARY

According to a preferred embodiment of the present invention for achieving the objects, a microfluidic device for studying shear stress and tumor migration in a microchannel includes: a microchamber; a microchannel including an inlet and an outlet for a fluid and formed on the periphery of the microchamber and making the fluid pass through the periphery of the microchamber; and a plurality of groups of bridge channels connecting the microchannel and the microchannel at a plurality of locations in the microchannel and a cross-sectional area of the bridge channel is smaller than that of the microchannel.

The microchamber may be positioned at the center and the microchannel may be formed while pivoting around the microchamber at the center and preferably, the microchannel may be formed in a circular shape and the microchamber may be positioned at the center.

The bridge channels of each group may be provided in the same number and the same size and a fluid moving distance (D) between the bridge channels of each group may be larger than a width (W) of the bridge channel of each group.

A plurality of microchambers may be provided around the inlet of the center, one end may be connected to the inlet of the center, the outlet may be formed at the other end, and portions connected to the bridge channel in the plurality of microchambers may be spaced from the microchannel by the same distance. The plurality of microchambers may be formed in an L shape, one end may be connected to the inlet of the center, a portion corresponding to the other side may be arranged in the microchannel in parallel and may be spaced apart by the same distance, and therefore, the microchannel may be formed in a circular shape.

Gelatin methacrylate (GelMA) having high biocompatibility may be provided to the microchannel and the bridge channel, the cell may be prevented from moving reversely by using the GelMA, and a specific factor is contained the GelMA to derive movement of the cell included in the microchannel.

According to a preferred embodiment of the present invention for achieving the objects of the present invention, a method for analyzing a change of a cell depending on shear stress by using a microfluidic device including a microchamber, a circular microchannel formed around the microchamber, and a plurality of groups of bridge channels connecting the circular microchannel and the microchamber at a plurality of locations in the circular microchannel includes: supplying a fluid including a cell to the circular microchannel at a predetermined flow velocity; and analyzing the cell at an inlet of the bridge channel connected to the circular microchannel, and a cross-sectional area of the bridge channel may be smaller than that of the circular microchannel.

A plurality of microchambers may be provided around the inlet of the center, one end may be connected to the inlet of the center, the outlet may be formed at the other end, and portions connected to the bridge channel in the plurality of microchambers may be spaced from the microchannel by the same distance.

The method may further include gelatin methacrylate (GelMA) having high biocompatibility is provided to the microchannel and the bridge channel to prevent the cell from moving to a cell chamber.

According to a preferred embodiment of the present invention for achieving the objects of the present invention, a method for analyzing movement of a cancer cell depending on shear stress by using a microfluidic device including a microchamber, a circular microchannel formed around the microchamber, and a plurality of groups of bridge channels connecting the circular microchannel and the microchamber at a plurality of locations in the circular microchannel includes: supplying a fluid including a cancer cell to the circular microchannel at a predetermined flow velocity; culturing the cancer cell in the circular microchannel; and analyzing that the cancer cell moves to the bridge channel connected to the circular microchannel and a cross-sectional area of the bridge channel may be smaller than that of the circular microchannel.

A plurality of microchambers may be provided around the inlet of the center, one end may be connected to the inlet of the center, the outlet may be formed at the other end, and portions connected to the bridge channel in the plurality of microchambers may be spaced from the microchannel by the same distance and gelatin methacrylate (GelMA) having high biocompatibility may be provided to the microchannel and the bridge channel and a vascular endothelial growth factor may be contained in the gelatin methacrylate to analyze the movement of the cancer cell in the bridge channel.

According to the present invention, it can be seen that a three-dimensional (3D) hydrogel based microfluidic device can be developed and optimized to study shear stress and cancer cell migration. In the microfluidic device, human umbilical vein endothelial cells (HUVECs) and breast cancer cell lines (MDA-MB-231) can be cultured and the effect of shear stress on the HUVECs in circular microfluidic channels can be investigated. As a result, it can be confirmed that the HUVEC exposed to the shear stress for 2 days in the circular microfluidic channel is elongated and aligned with a direction of a fluid and the breast cancer cell lines move to contained gelatin methacrylate (GelMA) hydrogel containing a vascular endothelial growth factor (VEGF). Therefore, this hydrogel based microfluidic device can be used as a useful tool for studying the shear stress and tumor migration applications.

### BRIEF DESCRIPTION OF DRAWINGS

FIG. 1 is a diagram for describing a microfluidic device for studying shear stress and tumor migration in a microchannel according to an embodiment of the present invention.

FIG. 2 is a diagram for describing a zone in the microfluidic device of FIG. 1.

FIG. 3 is a diagram for describing a fluorescence photograph of a microfluidic device for studying shear stress and tumor migration in a microchannel according to an embodiment of the present invention.

FIG. 4 is a diagram for describing a result of simulation by changing a height of a circular microchannel in a microfluidic device for studying shear stress and tumor migration in a microchannel according to an embodiment of the present invention.

FIG. 5 is a diagram for describing a result of simulation by changing a velocity of a fluid injected into a circular microchannel in a microfluidic device for studying shear stress and tumor migration in a microchannel according to an embodiment of the present invention.

FIG. 6 is a diagram for describing a confocal fluorescence microscope photograph of HUVEC cells cultured for 2 days in a microfluidic device according to an embodiment of the present invention.

FIG. 7 is a diagram for describing a graph for a survival rate of HUVEC cells exposed to a fluid flow for 2 days in a microfluidic device according to an embodiment of the present invention.

FIG. 8 is a diagram for describing a graph for an aspect ratio HUVEC cells exposed to a fluid flow for 2 days in a microfluidic device according to an embodiment of the present invention.

FIG. 9 is a diagram for describing a graph for a frequency of HUVEC cells exposed to a fluid flow for 2 days in a microfluidic device according to an embodiment of the present invention.

FIG. 10 is a diagram for describing a confocal fluorescence microscope photograph in a bridge channel in order to evaluate migration of metastatic breast cancer cells in a microfluidic device according to an embodiment of the present invention.

FIG. 11 is a graph for analyzing a graph for analyzing migration of metastatic breast cancer cells migrated to GelMA containing a vascular endothelial growth factor (VEGF) in order to evaluate the migration of the metastatic breast cancer cells in a microfluidic device according to an embodiment of the present invention.

### DETAILED DESCRIPTION

Hereinafter, embodiments of the present invention will be described in detail with reference to the accompanying drawings, but the present invention is not limited or restricted to the embodiments. For reference, in the description, like reference numerals substantially refer to like elements, which may be described by citing contents disclosed in other drawings under such a rule and contents determined to be apparent to those skilled in the art or repeated may be omitted.

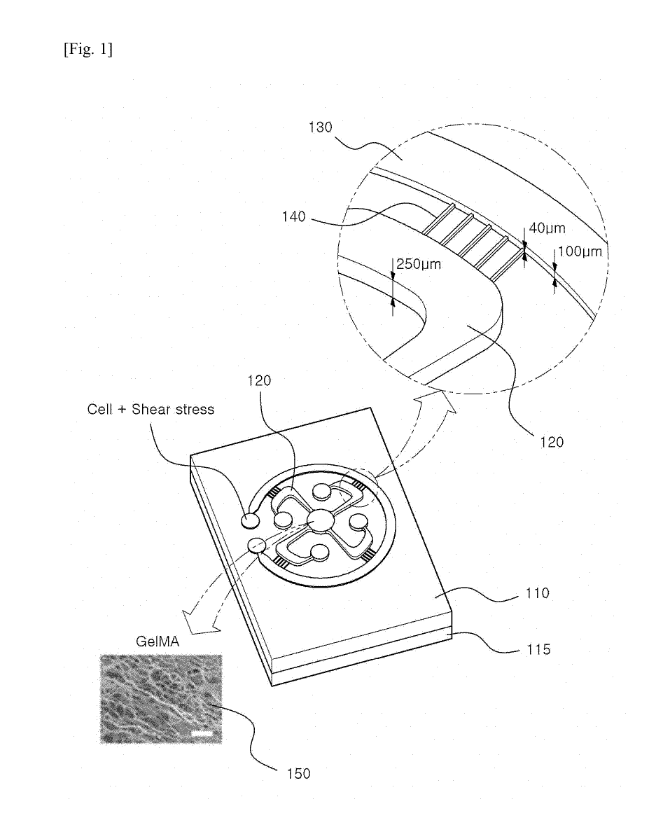

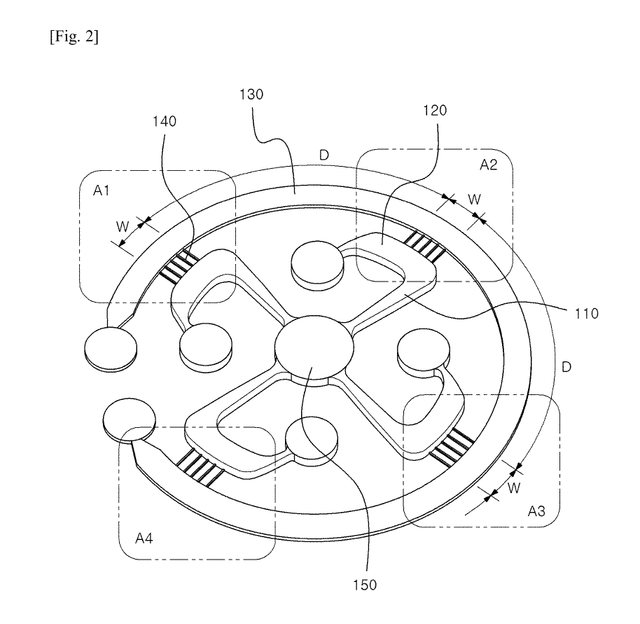

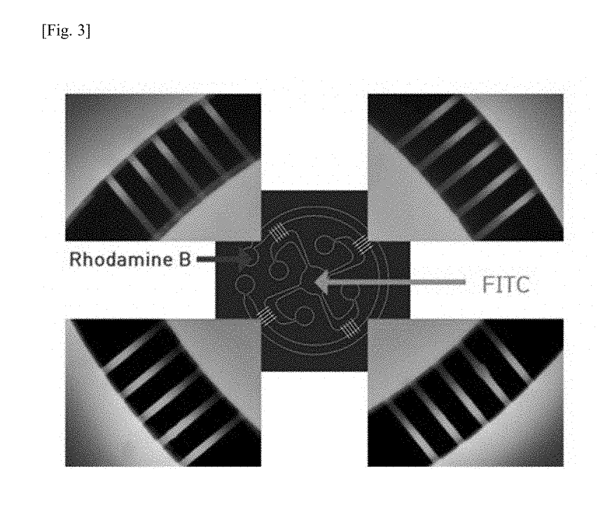

FIG. 1 is a diagram for describing a microfluidic device for studying shear stress and tumor migration in a microchannel according to an embodiment of the present invention, FIG. 2 is a diagram for describing a zone in the microfluidic device of FIG. 1, and FIG. 3 is a diagram for describing a fluorescence photograph of a microfluidic device for studying shear stress and tumor migration in a microchannel according to an embodiment of the present invention.

Referring to FIGS. 1 and 2, the microfluidic device 100 according to the embodiment may be formed using a multi-layer soft lithograph technique. The microfluidic device 100 includes a microchamber 120 located at the center of the microfluidic device 100, a circular microchannel 130 formed around the microchamber 120, and a bridge channel 140 connecting the microchamber 120 and the circular microchannel 130.

Here, the bridge channel 140 may be formed using a negative photoresist (PR) using SU-8. Specifically, after spin coating, the negative photoresist (PR) is exposed to ultraviolet light (UV) for approximately 60 seconds to form a silicon wafer 110 and a pattern for the bridge channel 140 may be formed on the silicon wafer in a thickness of approximately 40 μm.

On the silicon wafer for the bridge channel 140, the circular microchannel 130 may be formed in a thickness of approximately 100 μm using the negative photoresist using the SU-8 in the same method.

The microchamber 120 is formed in a thickness of approximately 250 μm on the silicon wafer in which the bridge channel 140 and the circular microchannel 130 are patterned by the same method to prepare the microfluidic device 100. In addition, a micromold may be made of poly(dimethyl siloxane) (PDMS) and a PDMS mold 115 may be subjected to a plasma treatment and then attached to the silicon wafer 110.

The fluid introduced into the circular microchannel 130 may be moved clockwise and discharged to an outlet and zones A1, A2, A3, and A4 may be set from an inlet. In the embodiment, the circular microchannel 130 may be divided into four zones, but the microchannel may be formed in a curved line or a straight line rather than a circle, and a plurality of zones other than four zones may be selected.

In the embodiment, in the case of the bridge channel 140, four groups may be formed in four zones and five bridge channels 140 may be provided in each group. The number of bridge channels 140 of each group may be preferably provided in the same number and the same dimension and a fluid movement distance D between the bridge channels 140 of each group may be larger than a width or a dimension W of the bridge channel of each group.

Further, in the microchamber 120 at the center, the fluid may be introduced from the center and the fluid may be supplied to the microchamber 120 corresponding to each bridge channel 140 and thereafter, discharged from an end of a chamber bent in an L shape to the outside again.

Referring to FIG. 3, it can be seen that a liquid mixed with rhodamine B is provided to the inlet of the circular microchannel 130 and a solution mixed with a fluorescent dye FITC is supplied to the microchamber 120.

Modeling for Shear Stress Gradients

A shear stress gradient may be modeled through a finite element analysis method by using the microfluidic device 100. On a simulation, a working fluid may be set to water, and since the working fluid is a flow in the microchannel, all the flows may be regarded as laminar and incompressible flows.

In the simulation, a Navier-Stokes equation and a continuity equation for analyzing a flow of a Newtonian fluid are used as governing equations for flow analysis as follows (see Equations 1 and 2).

$$\begin{matrix}ρ ( \frac{\partial u}{\partial t} + \overline{u} ( ∇ \cdot \overline{u} ) ) = - ∇ P + μ {∇}^{2} \overline{u} + g & [ Equation 1 ] \\ ∇ \cdot \overline{u} = 0 & [ Equation 2 ]\end{matrix}$$

Where u may represent a velocity vector, P may represent a pressure, g may represent a gravitational constant, and ρ and μ may represent a density and a viscosity of the fluid, respectively. According to normal water properties, ρ may 1,000 kg/m³ and μ, may be 1 mPas.

All walls of a model for the simulation are designated as non-slip boundaries, assuming a velocity of zero, and the fluid may be set so that the fluid exits only via a designated outlet and does not pass through the wall.

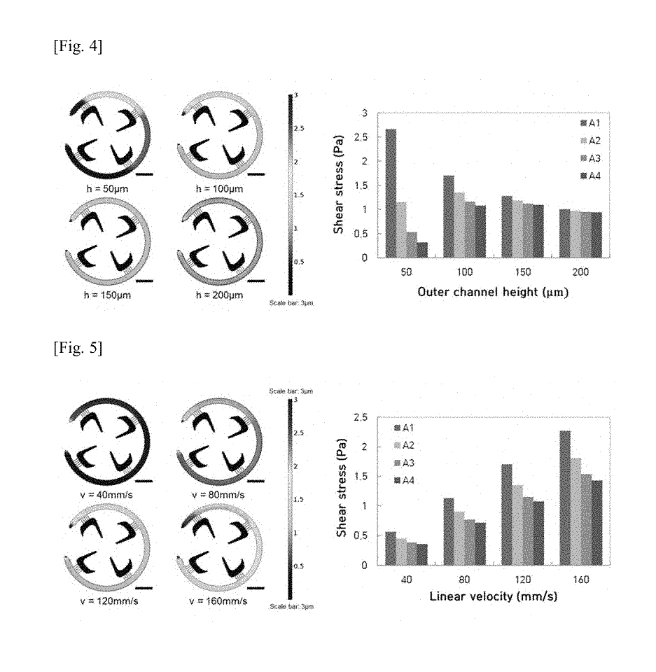

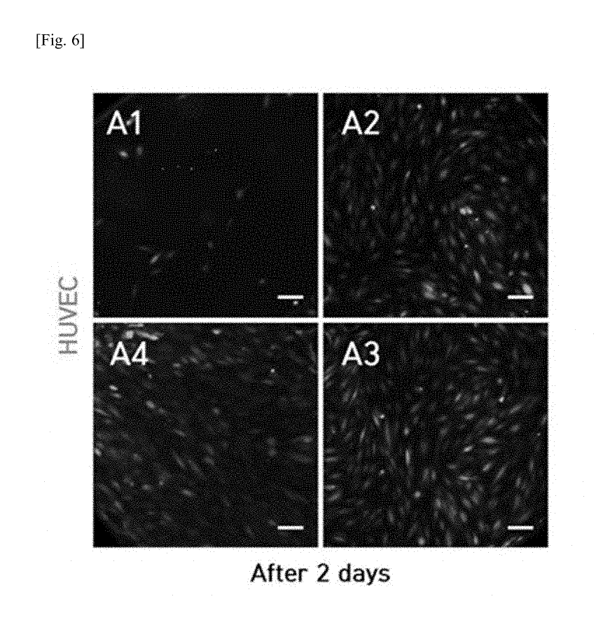

FIG. 4 is a diagram for describing a result of simulation by changing a height of a circular microchannel in a microfluidic device for studying shear stress and tumor migration in a microchannel according to an embodiment of the present invention and FIG. 5 is a diagram for describing a result of simulation by changing a velocity of a fluid injected into a circular microchannel in a microfluidic device for studying shear stress and tumor migration in a microchannel according to an embodiment of the present invention.

For reference, in FIG. 4, a result of performing the simulation is illustrated by changing a height of the circular microchannel 130 to 50, 100, 150, and 200 μm while fixing other dimensions and in FIG. 5, a result of performing the simulation is illustrated by changing a linear velocity of the fluid injected into the inlet to 40, 80, 120, and 160 mm/s while the height of the circular microchannel 130 to 100 μm. In this case, an outlet pressure is set to 0 in all simulations.

First, assuming that there is no bridge channel, as a simulation analysis result for the velocity and the shear stress, it can be seen that there is almost no change in velocity and shear stress in a θ direction in which the fluid flows. Here, a simplest method for changing the velocity is to change a cross-sectional area of the circular microchannel or an amount of the fluid flowing.

Referring to FIGS. 4 and 5, after the bridge channel 140 connecting the circular microchannel 130 and the microchamber 120 is added, the fluid moves from the circular microchannel 130 to the microchamber 120. The flow may reduce a flow velocity of the fluid flowing along the circular microchannel 130 in the θ direction, and cause the shear stress gradient in the microfluidic device 100.

Compared with a device without the bridge channel, it can be seen that the shear stress gradient is generated in the microfluidic device 100 including the bridge channel 140 through the simulation result.

In the embodiment, the same shear stress may be delivered in a section into which the bridge channel 140 is not inserted by locally making the bridge channel 140 without consecutively making the bridge channel in order to analyze how much the size of the shear stress influences the cell.

Since the bridge channel 140 is built in only an introduction portion of each of the areas A1 to A4, the flow rate in the microfluidic device 100 may be expressed by [Equation 4]. In addition, [Equation 5] and [Equation 6] may be introduced for the velocity and the shear stress, respectively through [Equation 4].

$$\begin{matrix}{Q}_{θ , i - 1} - {Q}_{θ , i} = {nQ}_{r} & [ Equation 4 ] \\ {υ}_{θ , i - 1} - {υ}_{θ , i} = \frac{{nA}_{r}}{{A}_{θ}} {υ}_{r} & [ Equation 5 ] \\ {τ}_{i - 1} - {τ}_{i} = \frac{{nA}_{r}}{{A}_{θ}} \frac{\partial {υ}_{r}}{\partial r} & [ Equation 6 ]\end{matrix}$$

Where n represents the number of bridge channels in each area, i represents numbers (0, 1, 2, 3, and 4) of the areas, Q_{θ,i} represents the flow rate in the circular microchannel in the i area in the θ direction, Qᵣ represents a flow rate which leaks to the bridge channel, and n represents the number of bridge channels.

[Equation 4] may be derived by conserving the flow rat and in [Equation 4], [Equation 5] may be obtained by dividing both sides by A_{θ} and Aᵣ. Here, A_{θ} and Aᵣ means cross-sectional areas of the circular microchannel and the bridge channel, respectively. Further, [Equation 6] summarized for the shear stress τ may be derived by differentiating both sides by r of [Equation 5].

According to an analytical solution described above, factors affecting the shear stress gradient may be expressed by the velocity, a cross-sectional ratio, and the number of bridge channels. Among them, an effect which the velocity and the cross-sectional ratio exerts may be analyzed through the simulation. Since the number (n) of bridge channels has a similar meaning as the cross-sectional ratio in terms of controlling a leakage amount of the fluid, the number (n) of bridge channels may not be considered.

As the simulation result according to the height of the circular microchannel 130, as illustrated in FIG. 4(b), the cross-sectional ratio of the bridge channel 140 to the circular microchannel 130 increases due to a small height and a difference in velocity between adjacent areas significantly occurs, and as a result, a larger shear stress gradient may be formed as the height of the circular microchannel 130 is smaller.

When only the shear stress gradient is considered, 50 m which is the smallest in height is suitable for comparison depending on the change, but when the height of the circular microchannel 130 shows a too small difference from the height of the bridge channel 140, the cell of the circular microchannel 130 may move very easily to the bridge channel 140 and the movement may generate unexpected variables. Therefore, in the simulation related to FIG. 5, the height of the circular microchannel 130 is set to 100 μm.

Referring to FIG. 5, the shear stress gradient according to a fluid injection velocity at the inlet may be analyzed. Referring to FIG. 5(b), it can be seen that the change in the linear velocity only changes an absolute magnitude of the shear stress, but the shear stress decreases at the same rate as in the previous area. However, when the fluid is injected at a too high velocity, since the cells of the circular microchannel 130 may not be settled and may flow together with the fluid, 120 mm/s may be selected at an optimum velocity.

Further, it can be discovered that the shear stress gradient is much more pronounced even when the circular microchannel 130 is changed in width of the channel in the θ direction. The above simulation model is implemented so that the width of a current area is doubled compared to the previous area. In the case of such a difference, it may be described that such a difference is generated due to an effect of (∂νᵣ/∂ᵣ) in which the fluid gets out to the bridge channel.

Moreover, according to [Equation 6], controlling (∂νᵣ/∂ᵣ) may also be a factor of shear stress gradient formation and in the microfluidic device used in the simulation, the cell may be prevented from moving to a cell chamber without restriction by using the GelMA (150 in the bridge channel. As a result, it may be anticipated that (∂νᵣ/∂ᵣ) is reduced and reduction of actual shear stress is smaller than the simulation.

As Gelma, biocompatible hydrogels synthesized with Type A Porcine skin gelatin and Methacrylate Anhydride may be used. When it is verified that GelMA mixed by inputting a photo initiator is injected into the inlet at the center of the microfluidic device 100 and is filled in the microchamber 120 and the bridge channel 140 and then, ultraviolet rays are irradiated, photo-crosslinking may be performed.

Analysis of Cell Deformation Using Microfluidic Device

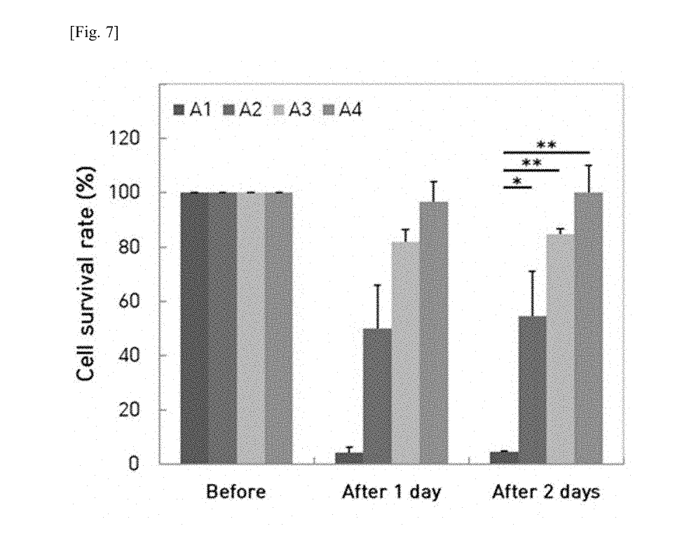

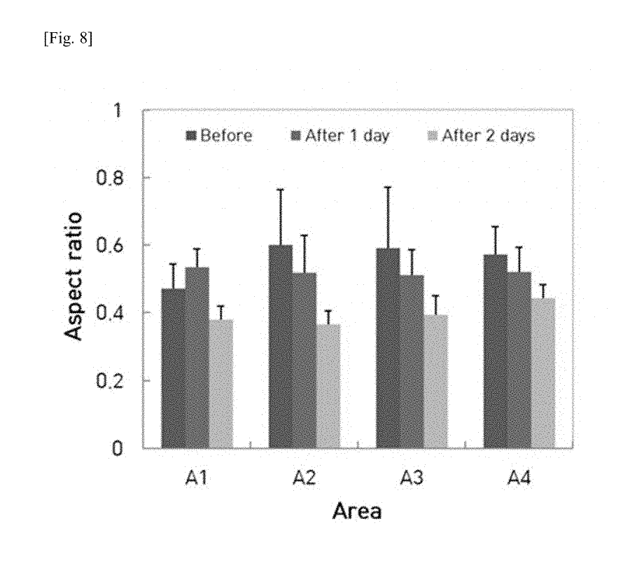

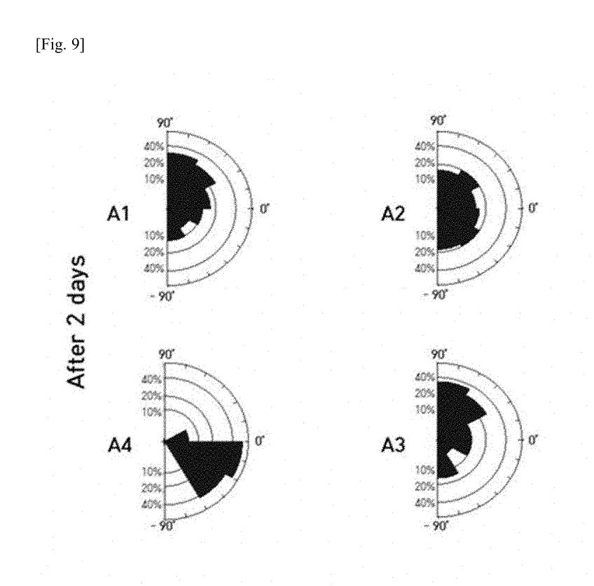

FIG. 6 is a diagram for describing a confocal fluorescence microscope photograph of HUVEC cells cultured for 2 days in a microfluidic device according to an embodiment of the present invention, FIG. 7 is a diagram for describing a graph for a survival rate of HUVEC cells exposed to a fluid flow for 2 days in a microfluidic device according to an embodiment of the present invention, FIG. 8 is a diagram for describing a graph for an aspect ratio HUVEC cells exposed to a fluid flow for 2 days in a microfluidic device according to an embodiment of the present invention, and FIG. 9 is a diagram for describing a graph for a frequency of HUVEC cells exposed to a fluid flow for 2 days in a microfluidic device according to an embodiment of the present invention.

Referring to FIGS. 6 to 9, the cells may be injected into the inlet of the circular microchannel 130 in the microfluidic device 100 filled with the GelMA. Human umbilical cord blood endothelial cells (HUVEC) were injected into the circular microchannel (130) in order to investigate the change of the cells according to the shear stress.

After confirming that the HUVEC cells were adhered 24 hours later, a cell culture solution was injected at a rate of approximately 120 mm/s using a syringe pump for 30 minutes for two days. The analysis was performed based on the vicinity of a location to which the bridge channel 140 was connected, and the areas A1, A2, A3, and A4 were analyzed from a range where the shear stress is the strongest. A cell survival rate may be confirmed for two days at four different shear stresses connected to the bridge channel 140.

Referring to FIG. 7, it can be confirmed that the survival rate in the A1 area was approximately 4.3% after one day and approximately 4.6% after two days, indicating that the survival rate was significantly reduced from day one. On the other hand, in the A4 area, the survival rate was approximately 96.8% after one day, and approximately 100.1% after two days, indicating that the survival rate again increased before the flow of the fluid again.

The change in the morphology of the cells according to the flow direction can be confirmed in the analysis range of the microfluidic device 100. Referring to FIG. 9, 26.3% of HUVEC cells were aligned at 31° to 60° in the A1 area, and 31.6% of HUVEC cells were aligned at 61° to 90°. 52.5% of HUVEC cells were aligned at 0° to (−29°) in the A4 area, and 42.5% of HUVEC cells were aligned at (−30°) to (−59°). HUVEC cells exposed to the shear stress depending on the flow are elongated and tend to align with a fluid direction.

Analysis of Cancer Cell Migration Using Microfluidic Device

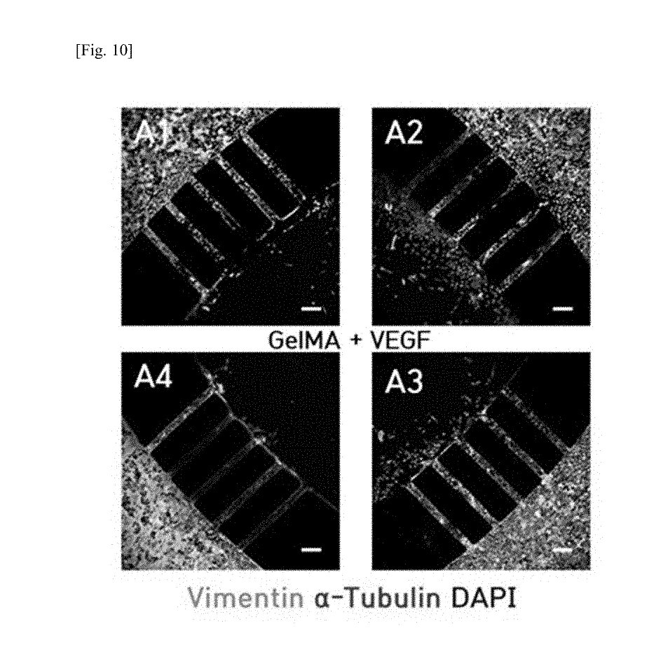

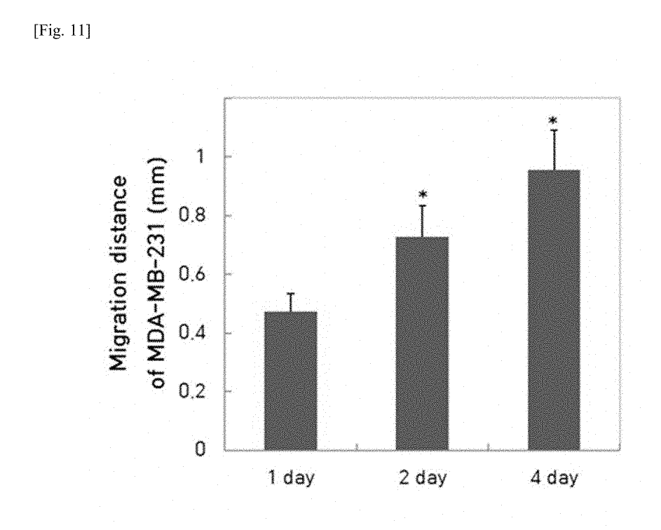

FIG. 10 is a diagram for describing a confocal fluorescence microscope photograph in a bridge channel in order to evaluate migration of metastatic breast cancer cells in a microfluidic device according to an embodiment of the present invention and FIG. 11 is a graph for analyzing a graph for analyzing migration of metastatic breast cancer cells migrated to GelMA containing a vascular endothelial growth factor (VEGF) in order to evaluate the migration of the metastatic breast cancer cells in a microfluidic device according to an embodiment of the present invention.

Referring to FIGS. 10 and 11, metastatic breast cancer cells (MDA-MB-231) were injected into the circular microchannel 130 to investigate a cell behavior in the microfluidic device 100. After 24 hours, MDA-MB-231 cells were confirmed to be adhered, and the vascular endothelial growth factor (VEGF) was added to the GelMA for 4 days. It can be seen that the MDA-MB-231 of the circular microchannel 130 moves to the fine chamber 120 with the GelMA containing the VEGF through the bridge channel 140. It can be seen that a total moving average distance of MDA-MB-231 cells after 4 days is approximately 950 μm.

According to the present invention, it can be seen that a three-dimensional (3D) hydrogel based microfluidic device can be developed and optimized to study shear stress and cancer cell migration. In the microfluidic device, human umbilical vein endothelial cells (HUVECs) and breast cancer cell lines (MDA-MB-231) can be cultured and the effect of shear stress on the HUVECs in circular microfluidic channels can be investigated. As a result, it can be confirmed that the HUVEC exposed to the shear stress for 2 days in the circular microfluidic channel is elongated and aligned with a direction of a fluid and the breast cancer cell lines move to contained gelatin methacrylate (GelMA) hydrogel containing a vascular endothelial growth factor (VEGF). Therefore, this hydrogel based microfluidic device can be used as a useful tool for studying the shear stress and tumor migration applications.

As described above, the present invention has been described with reference to the embodiments of the present invention. However, it will be appreciated by those skilled in the art that various modifications and changes of the present invention can be made without departing from the spirit and the scope of the present invention which are defined in the appended patent claims.

### DESCRIPTION OF REFERENCE NUMERALS

100: Microfluidic device
120: Microchannel
130: Circular microchannel
140: Bridge channel

What is claimed is:

1. A microfluidic device for studying shear stress and tumor migration in a microchannel, the device comprising:
a microchamber;
a microchannel including an inlet and an outlet for a fluid and formed on the periphery of the microchamber and making the fluid pass through the periphery of the microchamber; and
a plurality of groups of bridge channels connecting the microchannel and the microchannel at a plurality of locations in the microchannel,
wherein a cross-sectional area of the bridge channel is smaller than that of the microchannel.

2. The microfluidic device for studying shear stress and tumor migration in a microchannel of claim 1, wherein the microchamber is positioned at the center and the microchannel is formed while pivoting around the microchamber at the center.

3. The microfluidic device for studying shear stress and tumor migration in a microchannel of claim 2, wherein the microchannel is formed in a circular shape.

4. The microfluidic device for studying shear stress and tumor migration in a microchannel of claim 2, wherein the bridge channels of each group are provided in the same number and the same size and a fluid moving distance (D) between the bridge channels of each group is larger than a width (W) of the bridge channel of each group.

5. The microfluidic device for studying shear stress and tumor migration in a microchannel of claim 1, wherein a plurality of microchambers is provided around the inlet of the center, one end is connected to the inlet of the center, the outlet is formed at the other end, and portions connected to the bridge channel in the plurality of microchambers are spaced from the microchannel by the same distance.

6. The microfluidic device for studying shear stress and tumor migration in a microchannel of claim 5, wherein the plurality of microchambers is formed in an L shape.

7. The microfluidic device for studying shear stress and tumor migration in a microchannel of claim 5, wherein gelatin methacrylate (GelMA) having high biocompatibility is provided to the microchannel and the bridge channel.

8. A method for analyzing a change of a cell depending on shear stress by using a microfluidic device including a microchamber, a circular microchannel formed around the microchamber, and a plurality of groups of bridge channels connecting the circular microchannel and the microchamber at a plurality of locations in the circular microchannel, the method comprising:
supplying a fluid including a cell to the circular microchannel at a predetermined flow velocity; and
analyzing the cell at an inlet of the bridge channel connected to the circular microchannel,
wherein a cross-sectional area of the bridge channel is smaller than that of the circular microchannel.

9. The method of claim 8, wherein a plurality of microchambers is provided around the inlet of the center, one end is connected to the inlet of the center, the outlet is formed at the other end, and portions connected to the bridge channel in the plurality of microchambers are spaced from the microchannel by the same distance.

10. The method of claim 8, wherein gelatin methacrylate (GelMA) having high biocompatibility is provided to the microchannel and the bridge channel to prevent the cell from moving to a cell chamber.

11. The method of claim 8, the method further comprising:
culturing the cell in the circular microchannel.

12. A method for analyzing movement of a cancer cell depending on shear stress by using a microfluidic device including a microchamber, a circular microchannel formed around the microchamber, and a plurality of groups of bridge channels connecting the circular microchannel and the microchamber at a plurality of locations in the circular microchannel, the method comprising:
supplying a fluid including a cancer cell to the circular microchannel at a predetermined flow velocity;
culturing the cancer cell in the circular microchannel; and
analyzing that the cancer cell moves to the bridge channel connected to the circular microchannel,
wherein a cross-sectional area of the bridge channel is smaller than that of the circular microchannel.

13. The method of claim 12, wherein a plurality of microchambers is provided around the inlet of the center, one end is connected to the inlet of the center, the outlet is formed at the other end, and portions connected to the bridge channel in the plurality of microchambers are spaced from the microchannel by the same distance.

14. The method of claim 12, wherein gelatin methacrylate (GelMA) having high biocompatibility is provided to the microchannel and the bridge channel and a vascular endothelial growth factor is contained in the gelatin methacrylate to analyze the movement of the cancer cell in the bridge channel.
